(19) United States
(12) Patent Application Publication
(10) Pub. No.: US 2005/0080488 A1
(43) Pub. Date: Apr. 14, 2005

(54) Intervertebral implant

(76) Inventor: Robert Schultz, Tuttlingen (DE)

Correspondence Address: RATNERPRESTIA, P O BOX 980, VALLEY FORGE, PA, 19482-0980, US

(21) Appl. No.: 10/882,618

(22) Filed: Jun. 30, 2004

(30) Foreign Application Priority Data
Oct. 8, 2003 (DE) 103 47 172.3

(51) Int. Cl. A61F 2/44

(52) U.S. Cl. 623/17.13; 623/17.14

(57) ABSTRACT

An intervertebral implant is provided for insertion between a first vertebral body and a second vertebral body defining an intervertebral space. The implant includes a first anchoring part for anchoring at the first vertebral body, a second anchoring part for anchoring at the second vertebral body, and a joint configured to connect the first and second anchoring parts together. The joint includes a first joint part carried by the first anchoring part, and a second joint part carried by the second anchoring part. The implant further includes at least one restoring device adapted to exert a restoring force on the first or second joint part. The restoring force of the at least one restoring device transfers the first or second joint part from a deflected position back to a normal position and/or limits movement of the first or second joint part away from the normal position.

This application is related to and claims the benefit of German Utility Model No. 203 15 611.0 entitled Intervertebral Implant issued on Dec. 11, 2003, and German Patent Application No. 103 47 172.3 filed Oct. 8, 2003.

### FIELD OF THE INVENTION

The present invention pertains to an intervertebral implant with which the original height of the intervertebral disk can be restored in case of, e.g., degeneratively altered intervertebral disks, and the function can be preserved at the same time.

### BACKGROUND OF THE INVENTION

Intervertebral implants are used, among other things, in the case of degeneratively altered intervertebral disks as the replacement thereof in order to restore an original intervertebral disk height while preserving the function at the same time. Numerous embodiments of intervertebral implants in the form of intervertebral disk prostheses are known, and most of the prostheses used clinically are based on the principle of the ball and socket joint, in which the two joint parts are designed such that they form together a ball and socket joint with a center of rotation. Based on intervertebral disk prostheses that have a rotation center which is fixed in relation to the two anchoring parts, intervertebral disk prostheses have been proposed that make possible the movement of one of the two joint parts in relation to one of the anchoring parts. An example of such intervertebral disk prostheses is known from FR 2 730 159, in which one of the joint parts is mounted on a convexly curved bearing surface, i.e., on a movement surface, in a horizontal plane extending at right angles or essentially at right angles to a connection direction of the two joint parts. A movement surface is defined in terms of this application as all surfaces that extend flatly, e.g., in parallel to a horizontal plane, or are not completely flat, and, in particular, also curved, for example, concave or convex surfaces.

The drawback of the prior-art intervertebral disk prostheses with at least one joint part mounted movably in relation to the corresponding anchoring part is that the joint part is completely freely movable and may strike the edges of a recess in the anchoring part and may be damaged as a result.

Accordingly, there remains a need for an improved intervertebral implant such that the most natural mobility possible of the spinal column can be restored and the implant has a long life at the same time.

### SUMMARY OF THE INVENTION

The present invention pertains to an intervertebral implant for insertion between a first and a second vertebral body defining an intervertebral space, comprising a first anchoring part for anchoring at the first vertebral body, a second anchoring part for anchoring at the second vertebral body, and a joint connecting the first and second anchoring parts and comprising first and second joint parts. The first anchoring part carries the first joint part and the second anchoring part carries the second joint part, wherein the first and/or second joint part is mounted at the corresponding anchoring part such that it is movable on a movement surface extending at right angles or essentially at right angles to a connection direction of the two joint parts and can be brought from a normal position into a deflected joint part position.

At least one restoring device is designed such that it exerts a restoring force on the first or second joint part deflected from the normal position to transfer the first or second joint part from the deflected position of the joint part back into the normal position and/or to limit a movement of the first or second joint part away from the normal position.

For example, the displaceably mounted bearing part known from FR 2 730 159 can be effectively prevented, with the restoring device of the present invention, from being able to strike lateral edges of the anchoring part and being damaged as a result. Undesired abrasion is thus avoided and the life of the implant as a whole is prolonged. Moreover, the design supports the maintenance of the intervertebral implant in its normal position. The restoring device of the present invention promotes the stability of the intervertebral disk, especially in patients who have weakened muscles. On the whole, the natural mobility is reconstructed nearly optimally with the intervertebral implant according to the present invention because, just as in the natural intervertebral disk, a translational motion of a joint center in parallel to the plane of motion is damped.

It is favorable for the at least one restoring device to comprise at least one restoring element for transferring one of the two joint parts from the deflected position of the joint part back into the normal position and/or for limiting a movement of one of the two joint parts away from the normal position. Depending on the desired mobility of the implant, one or more restoring elements may be used in order to limit or force the movement of at least one of the two joint parts on the movement surface in the desired manner. Identical or different restoring elements may be used. The restoring device may also comprise a carrier element, which carries the at least one restoring element.

The joint is preferably designed such that it permits rotary movements of the two anchoring parts in relation to one another around three linearly independent space axes. Optimal mobility of the intervertebral implant can thus be achieved and mobility of the spinal column can be restored, on the whole, in the original form.

The design of the implant becomes even simpler when the joint is a ball and socket joint. For example, a ball and socket joint can be formed in a simple manner by a convexly curved spherical bearing surface and a concavely curved hollow spherical bearing surface.

It is especially advantageous if the at least one restoring element is elastic. This enables the restoring element to resume its original shape after a deflection or deformation, as a result of which the life of the implant is prolonged.

It is advantageous if tensile forces can be exerted with the at least one restoring device on the first or second joint part. The joint element deflected from the normal position can thus be pulled back into the normal position. For example, joint elements can be pulled back into the normal position with coil springs or the like.

The at least one restoring device is preferably connected with the first anchoring part and the first joint part or with the second anchoring part and the second joint part. The at least one restoring device can thus return the first or second joint part into the normal position when these joint parts are deflected or hold them in the normal position. Furthermore, separation of the joint part from the anchoring part is not possible, because the at least one restoring device connects the anchoring part with the corresponding joint part belonging to it.

Furthermore, it may be advantageous if compressive forces can be exerted with the at least one restoring device on the first joint part or the second joint part. The advantage of this embodiment is that a connection is not absolutely necessary between the joint part and the anchoring part. For example, a plurality of restoring elements acting independently from one another may exert compressive forces on one of the two joint parts.

The design of the implant becomes simplified if at least one of the two anchoring parts has at least one stop acting at right angles to the movement surface and if the at least one restoring device is supported at the first joint part and at the at least one stop of the first anchoring part and/or at the second joint part and at the at least one stop of the second anchoring part. The stop can absorb, in principle, both compressive forces and tensile forces, which can be exerted by the restoring device, in order to hold one or both of the joint parts in the normal position or to limit the movement of that joint part/those joint parts.

The design becomes especially simple if at least one of the two anchoring parts has a stop surrounding the corresponding joint part at a spaced location. The movement of the at least one joint part in a direction at right angles to the connection direction is limited as a result, for example, on the movement surface. In addition, the restoring device can thus damp on all sides a deflecting movement of one of the two joint parts on the movement surface from the normal position.

It is favorable for at least one of the two anchoring parts to have a joint part mount for receiving the corresponding joint part at least partially. This mount is preferably designed such that the joint part has a certain freedom of movement in all directions on the movement surface or is guided along a linear or curved guideway.

It is advantageous if the joint part mount of one of the two anchoring parts has a surrounding edge or a surrounding wall and if the edge or the wall forms the at least one stop. The design of the implant is thus additionally simplified. In addition, the overall height of the anchoring parts can be minimized.

According to a preferred embodiment of the present invention, provisions may be made for at least one of the two joint parts to be able to be detachably connected with the corresponding anchoring part. This is especially favorable if one of the two joint parts is to be mounted immovably in relation to the corresponding anchoring part. Moreover, the joint parts can be replaced in a simple manner in this case, which could be necessary, for example, because of wear. In addition, joint parts can be replaced even during an operation and higher or flatter joint parts can be inserted as needed in order to reconstruct the intervertebral space in the original shape and height.

It is favorable if at least one joint part mount is designed such that the particular joint part is held in a positive-locking and/or nonpositive manner in the at least one joint part mount. An unintended separation of the joint part from the corresponding anchoring part is prevented from occurring as a result. In addition, a positive-locking and/or nonpositive connection can be established in an especially simple manner.

To ensure the free mobility of the joint part in the joint part mount, it is advantageous if at least one joint part mount has a larger cross section than the joint part immersing into the at least one joint part mount and is designed such that the joint part is displaceable on the movement surface. A joint center can thus be moved, for example, along a path that is parallel to the movement surface, which corresponds to the natural motion characteristic of an intervertebral disk.

It is advantageous if the at least one joint part mount has a larger cross section than the joint part immersing into the at least one joint part mount and is designed such that the joint part is displaceable along a straight or curved guideway on the movement surface. This embodiment is desirable if a more or less free mobility of the joint part in the horizontal plane is not desired in certain patients, but an exactly defined mobility along a guideway is desired. The guideway may extend in a horizontal plane or also be curved out of such a horizontal plane.

To improve the cohesion of the implant, it may be favorable if the at least one restoring element is designed such that it is held in the at least one joint part mount in a positive-locking or nonpositive manner. It is ensured as a result that the restoring element is held captively at the at least one anchoring part and cannot be moved out of the joint part mount even under a heavy load and the action of strong forces due to the joint part.

It may be advantageous, in principle, for the at least one restoring element to be elastic. This makes it possible to use conventional, commercially available elastic elements, for example, coil springs, as restoring elements.

A restoring element can be manufactured in different shapes in an especially simple manner if it is made of a plastic. In addition, it is especially lightweight and may also have an especially high abrasion resistance depending on the plastic selected.

Provisions may be made according to a preferred embodiment of the present invention for the at least one restoring element to have an essentially annular design. This makes it possible for the restoring element to surround at least one of the two joint parts in an annular manner. However, it is also possible that the annular restoring element is laterally in contact with one of the two joint parts and thus forms an annular spring, whose diameter is changed by the action of an external force.

It is advantageous for the at least one restoring element to surround one of the two joint parts in a positive-locking manner. A deflecting motion of the joint part from the normal position can thus be damped especially softly. In addition, the hold of the joint part at the anchoring part is additionally increased.

The restoring elements may have, in principle, many different shapes. The at least one restoring element is preferably designed in the form of a leaf spring curved convexly toward at least one of the two joint parts. Such restoring elements can be manufactured in an especially simple manner and at an especially low cost.

In order to advantageously minimize and preset a damping path for a deflecting motion of the joint element, the at least one restoring element may be a leaf spring of a snake-like shape.

It would, in principle, be possible and conceivable for the restoring device to exert a force on the joint part assuming the normal position. To increase the hold of the joint part and the stability of the implant, it may, however, be desirable for the restoring device to hold at least one of the two joint parts in a pretensioning manner in the normal position. Deflecting forces overcoming the pretensioning force must thus be exerted in order to bring about a deflection of the joint part from the normal position in the first place. This may be desirable especially in case of large and robust patients.

It may be advantageous to provide a plurality of restoring elements and to arrange the restoring elements symmetrically around at least one of the two joint parts. This makes it possible to design the restoring device as a whole such that it can assume the natural function of the intervertebral disk nearly identically. The elastic properties of restoring elements may be made equal or different, so that, for example, deflections in different directions on the movement surface are possible with different degrees of difficulty.

It is advantageous to provide for the implant a set of restoring devices with different elasticities, which can be used alternatively. This makes it possible to form implants tailored especially to one patient by using the most suitable restoring device depending on the orthopedic situation, be it in terms of the height of the individual anchoring site and consequently of the implant as a whole or also in terms of the lateral forces acting in parallel to the movement surface, which are to be absorbed by the implant.

An especially good joint can be designed in a simple manner by the first joint part having a convexly curved first bearing surface and by the second joint part having a concavely curved bearing surface. For example, it is thus possible to design ball and socket joints or, if the radii of curvature of the two joint parts are not identical, to make additionally possible a translational motion of the two joint parts in relation to one another.

It is advantageous for the revision of the implant if the restoring device is replaceable. This also makes it possible to select and insert a restoring device that is optimal for the patient after or during a surgical procedure performed to replace a natural, degenerated intervertebral disk.

It is possible, in principle, to use the intervertebral implant as such purely to replace a natural, degenerated intervertebral disk. However, provisions shall also be made according to a preferred embodiment of the present invention for at least one of the two anchoring parts to be able to be connected with a vertebral body replacement implant. This makes it possible to replace a degenerated vertebral body of the human spinal column and to directly connect an anchoring part of the intervertebral implant with the vertebral body replacement implant. Practically all orthopedically conceivable cases can thus be treated for the reconstruction of a spinal column.

It is advantageous if at least one of the two anchoring parts and/or the corresponding joint part is provided with a wear-reducing coating, which is designed such that the coating forms a contact surface between at least one of the two anchoring parts and the corresponding joint part. Maintenance or revision of the implant becomes necessary only markedly later due to the special coating than it would without such a coating. In particular, the sometimes very complicated coating is simplified if the joint part is provided with the coating because, on the whole, a smaller area needs to be coated than in the case of a bearing surface of one of the two anchoring parts.

It is favorable if at least one of the two anchoring parts and/or at least one of the two joint parts is made of a material that is physiologically compatible. Rejection reactions by the human body are thus prevented from occurring.

The first anchoring part, the second anchoring part, the first joint part, and the second joint part are preferably made of the same material or different materials which are physiologically compatible. Depending on the function, the individual parts of the implant may be made individually from different materials or also from the same materials. The selection depends essentially on what function the individual parts must assume, i.e., whether they assume a joint function or a carrying function.

It is favorable if the physiologically compatible material is a metal, a ceramic or a plastic. The materials, in particular, metal or plastic, can be processed in a simple manner, and, moreover, ceramic has a high abrasion resistance, which is especially well suited for forming surfaces of joint parts or joint parts as a whole.

### BRIEF DESCRIPTION OF THE DRAWINGS

FIG. 1 shows a longitudinal sectional view of an intervertebral disk prosthesis according to the present invention inserted between two vertebral bodies;

FIG. 2 shows a top view of a base plate of the intervertebral disk prosthesis shown in FIG. 1;

FIG. 3 shows a sectional view along line 3-3 in FIG. 2;

FIG. 4 shows a top view of a base plate of a second exemplary embodiment of an intervertebral disk prosthesis;

FIG. 5 shows a sectional view along line 5-5 in FIG. 4;

FIG. 6 shows a top view of a base plate of a third exemplary embodiment of an intervertebral disk prosthesis according to the present invention;

FIG. 7 shows a sectional view along line 7-7 in FIG. 6;

FIG. 8 shows a top view of a base plate of a fourth exemplary embodiment of an intervertebral disk prosthesis according to the present invention;

FIG. 9 shows a sectional view along line 9-9 in FIG. 8;

FIG. 10 shows a top view of a base plate of a fifth exemplary embodiment of an intervertebral disk prosthesis according to the present invention; and

FIG. 11 shows a top view of a base plate of a sixth exemplary embodiment of an intervertebral disk prosthesis according to the present invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

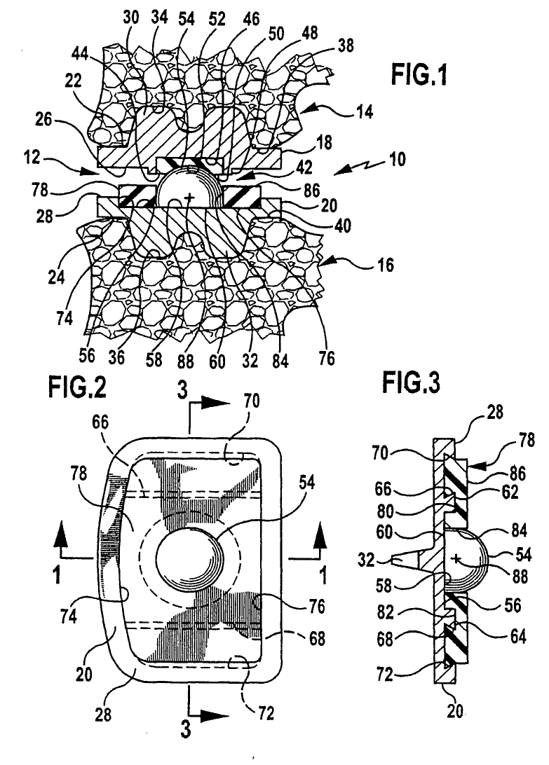

FIG. 1 shows an intervertebral disk prosthesis designated as a whole by the reference number 10. It is inserted in an intervertebral space 12 between a first vertebral body 14 and a second vertebral body 16.

The intervertebral disk prosthesis 10 as a whole has an essentially two-part design and comprises a first base plate 18 and a second base plate 20, which comprise a respective anchoring surface 22 and 24 each and a respective bearing surface 26 and 28 each. Narrow, plate-like anchoring ribs 30 and 32, which are driven into the vertebral bodies 14 and 16 to anchor the base plates 18 and 20 or are inserted into respective recesses 34 and 36 prepared for this purpose, project at right angles from the anchoring surfaces 22 and 24. The anchoring surfaces 22 and 24 are substantially two-dimensionally in contact with the respective surfaces 38 and 40 of the respective vertebral bodies 14 and 16, which point toward each other and define the intervertebral space 12 between them. The shape of the anchoring surfaces 22 and 24 is selected essentially corresponding to the shape of the surfaces 38 and 40 of the vertebral bodies 14 and 16, so that the greatest possible overlap of the anchoring surfaces 22 and 24 and the surfaces 38 and 40 is achieved.

The intervertebral disk prosthesis 10 comprises, furthermore, a joint 42, which is formed by a bearing plate 44 inserted into a recess 46 in the first base plate 18. The recess 46 is surrounded by an edge 48 projecting at right angles from the bearing surface 26, so that a surface 50 of the bearing plate 44 facing the direction of the second base plate 20 ends flush with the edge 48. The bearing plate 44 also fills out the recess 46 in a positive-locking manner. The bearing plate 44 forms a first joint part due to the fact that it is provided with a concave hollow spherical bearing surface 52, which faces the direction of the second base plate 20.

A second joint part of the joint 42 is formed by a hemispherical sliding body 54, which has a radius of curvature that corresponds to the bearing surface 52. A ball and socket joint is thus formed.

The base plate 20 is provided with a substantially rectangular depression 56, which comprises a flat sliding surface 58 facing the first base plate 18. The sliding body 54 has a flat sliding surface 60, which lies directly on the sliding bearing surface 58 forming a movement surface. The dimensions of the depression 56 are selected to be such that the sliding body 54 can slide on the sliding bearing surface 58 in all directions in parallel to that sliding bearing surface 58. The sliding bearing surface 58 and/or the sliding surface 60 may be optionally provided with a wear-reducing coating.

Two holding ribs 62 and 64, shown in FIG. 3, are formed in the depression 56 extending symmetrically and in parallel to the anchoring rib 32, projecting in the direction of the first base plate 18. A respective lateral edge 66 and 68 of the holding ribs 62 and 64 is undercut. Furthermore, edges 70 and 72 of the depression 56, which extend laterally in parallel to the holding ribs 62 and 64, are likewise undercut. The depression 56 is limited forward and backward by a slightly convexly curved front edge 74 and by a straight rear edge 76. The depression 56 is filled out in a positive-locking manner by a damping element 78, which has two longitudinal recesses 80 and 82, which correspond to the holding ribs 62 and 64 and are filled out by the holding ribs 62 and 64 in a positive-locking manner. An opening, which forms a sliding body mount 84, is provided approximately in the middle of the damping element 78. The damping element 78 has a thickness that is approximately twice the depth of the depression 56, so that a damping element surface 86, which faces the first base plate 18, projects somewhat over the bearing surface 28.

The sliding body mount 84 has a round cross section, which is adapted to the diameter of the sliding body 54, so that this is surrounded by the damping element 78 in an annular manner.

The intervertebral disk prosthesis 10 shown in FIGS. 1 through 3 is consequently designed such that a rotation center 88 of the joint 42 in relation to the second base plate 20 is freely movable, substantially in parallel to the sliding bearing surface 58. Deflection of the sliding body 54 from the symmetrical normal position shown in FIGS. 1 thorough 3 is damped by the damping element 78, which is made of an elastic material, for example, an elastomer. If the sliding body 54 is deflected from the normal position, the damping element 78 exerts a restoring force on the sliding body 54 against the direction of deflection, so that the sliding body 54 is again returned into the normal position. Furthermore, the holding ribs 62 and 64 form stops, at which the damping element 78 can be supported. This also applies to the edges 74 and 76. On the whole, the intervertebral disk prosthesis 10 imitates the function of a natural intervertebral disk in a nearly ideal manner.

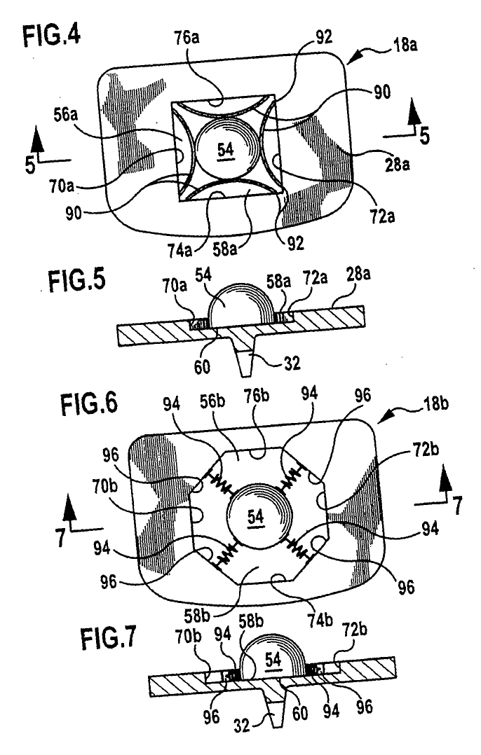

FIGS. 4 through 11 show additional exemplary embodiments of the intervertebral disk prostheses according to the present invention. Their basic design corresponds to that of the intervertebral disk prosthesis 10. Thus, all other exemplary embodiments are provided with a base plate 18, which has a design identical to that of the prosthesis 10 described above with reference to FIGS. 1 through 3. Therefore, only the differences from the intervertebral disk prosthesis 10, which are limited to the form of the mounting of the sliding body 54 on the second base plate 20, will be discussed below. Identical or very similar parts of the described exemplary embodiments of the intervertebral disk prostheses are therefore designated by the same reference numbers for the sake of clarity.

Another variant of the base plate 18 of the intervertebral disk prosthesis 10 is designated by reference number 18a. FIG. 4 shows a top view of the first base plate 18a, which has a square depression 56a in the bearing surface 28a, the depression 56a being limited laterally by the edges 70a, 72a, 74a, and 76a. A sliding bearing surface 58a of the depression 56a, which faces the second base plate 20 (not shown), is designed as a flat surface. The sliding surface 60 of the sliding body 54 rests on the sliding bearing surface 58a. To hold the sliding body 54 in the normal position, four identical leaf springs 90 are provided, which are curved convexly in the direction of the sliding body 54 and whose free ends 92 are supported at the edges 70a, 72a, 74a, and 76a. The leaf springs 90 thus limit a deflecting movement of the sliding body 54 within the depression 56a in the direction of the edges 70a, 72a, 74a, and 76a. Furthermore, the leaf springs 90 exert a force on the sliding body 54, which returns the sliding body 54 in the direction of its normal position, which normal position is shown in FIG. 4.

Yet another alternative of a base plate 18b, shown in FIGS. 6 and 7, differs from the base plate 18a in that the depression 56b of base plate 18b is octagonal.

Instead of the leaf springs 90, four identical coil springs 94 are provided, which are fastened to the sliding body 54 at edge surfaces 96. The coil springs 94 exert tensile forces and compressive forces on a sliding body 54 deflected from the normal position shown in FIG. 6 in order to damp the deflecting movement and to return the sliding body 54 into the normal position, in which it is positioned centrally within the depression 56b.

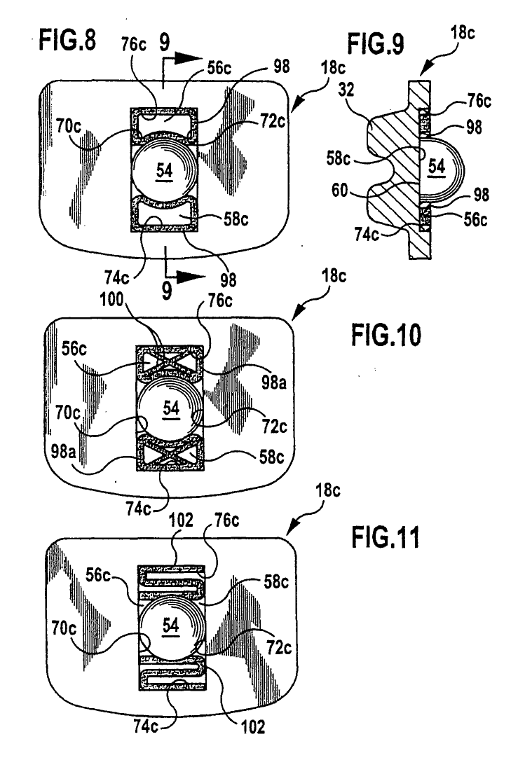

Another variant of a base plate 18c is shown in FIGS. 8 through 11. It comprises a rectangular depression 56c, which is extended in an oblong pattern in parallel to the anchoring rib 32 and whose width corresponds to the diameter of the sliding body 54. A linear guideway is formed for the sliding body 54 as a result. Two damping rings 98 damp a deflecting movement in the direction of the edges 74c and 76c of the depression 56c. The damping rings 98 are formed essentially from an elastic material, which conforms to the edges 70c, 72c, 76c of the depression 56c for one damping ring 98 on one end, edges 70c, 72c, and 74c for another damping ring 98 on the other end, as well as the sliding body 54.

FIG. 10 shows the base plate 18c with a modified damping ring 98a, which forms a variant of the damping ring 98 and has, in addition, two webs 100, which cross each other within the ring structure. The elasticity of the damping ring 98a is reduced by the webs 100 compared with the damping ring 98, providing that both damping rings are made of the same elastic material.

As an alternative to the damping rings 98 and 98a, a restoring element in the form of an S spring 102 is inserted into the depression 56c, as shown in FIG. 11, between the sliding body 54 and the edge 76c of the depression 56c on one side, and an S spring 102 is inserted between the sliding body 54 and the edge 74c on the other side. Due to its S shape, the S spring 102 is further supported at mutually opposite edges 70c and 72c of the depression 56c.

Biocompatible metals, especially titanium alloys or chromium-cobalt alloys, are preferably used as the material for all of the above-described base plates 18 through 18c. As explained above, the sliding bearing surface 58 is preferably provided with a wear-reducing coating, which helps avoid an unintended abrasion between the sliding body 54 and the base plate 18. The sliding body 54 and the bearing plate 44 may be made of a ceramic material. As an alternative, sliding bodies 54 and bearing plates 44 made of plastic, especially from PEEK™, which is a polymer (polyether ether ketone) manufactured by Victrex® PLC of the United Kingdom. PEEK™ is transparent to X-rays, which leads to a great advantage in postoperative X-ray diagnostics with CTs or nuclear spin tomography, because, unlike metals, the plastic does not cause any artifacts (i.e., obstructions) in the X-ray image.

The ceramic components (sliding body 54 and the bearing plate 44) are manufactured with corresponding precision such that the wear such a ball and socket joint 42 nearly equals zero. A further advantage of the ceramic-on-ceramic bearing is that the problem of creep under load, which is peculiar to polyethylene, is absent. Since the ceramic material of the sliding body 54 and the bearing plate 44 has a substantially higher compressive strength and dimensional stability than polyethylene, the dimensions of joint 42 may be reduced. The forced translational motion superimposed to the flexion/extension movement decreases as a result.

All the restoring elements described above, namely, the damping element 78, the leaf springs 90, the coil springs 94, the damping rings 98 and 98a, and the S spring 102 may be made of plastic, and all restoring elements except for the damping element 78 may be optionally made of a metal. Different elastic properties may be achieved either by means of plastics of different hardness or by making the elements from the same material, but affecting the elasticity by design (e.g., different wall thicknesses).

The intervertebral disk prosthesis 10 can be inserted with the aid of navigated instruments. In use, the ceramic sliding body 54 and bearing plate 44 are inserted into the base plates 18 through 18c and 20 prior to implantation, and the intervertebral disk prosthesis 10 is implanted in the assembled state, thereby significantly simplifying the implantation procedure.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. An intervertebral implant for insertion between a first vertebral body and a second vertebral body defining an intervertebral space, said implant comprising:
a first anchoring part for anchoring at the first vertebral body;
a second anchoring part for anchoring at the second vertebral body;
a joint configured to connect said first and second anchoring parts together, said joint comprising
a first joint part carried by said first anchoring part, and
a second joint part carried by said second anchoring part; and
at least one restoring device adapted to exert a restoring force on said first or second joint part,
wherein said first and/or said second joint part is mounted at said corresponding first and/or second anchoring part such that it is movable on a movement surface extending substantially perpendicular to a connection direction of said first and second joint parts and can be brought from a normal joint position into a deflected joint position,
and wherein said restoring force of said at least one restoring device transfers said first or second joint part from said deflected position back to said normal position and/or limits movement of said first or second joint part away from said normal position.

2. The implant of claim 1, wherein said at least one restoring device comprises at least one restoring element for transferring one of said two joint parts from said deflected position of said joint part back to said normal position and/or for limiting movement of one of said two joint parts away from said normal position.

3. The implant of claim 1, wherein said joint is configured to permit rotary movement of said first and second anchoring parts in relation to one another around three linearly independent space axes.

4. The implant of claim 1, wherein said joint is a ball and socket joint.

5. The implant of claims 2, wherein said at least one restoring element has elastic properties.

6. The implant of claim 1, said at least one restoring device exerts tensile forces on said first or said second joint part.

7. The implant of claim 1, wherein said at least one restoring device is connected with said first anchoring part and with said first joint part or with said second anchoring part and said second joint part.

8. The implant of claim 1, said at least one restoring device exerts compressive forces on said first joint part or said second joint part.

9. The implant of claim 1, wherein at least one of said first and second anchoring parts comprises at least one stop acting substantially perpendicular to said movement surface and said at least one restoring device is supported at said first joint part and at said at least one stop of said first anchoring part and/or at said second joint part and at said least one stop of said second anchoring part.

10. The implant of claim 9, wherein at least one of said first and second anchoring parts comprises said stop surrounding said corresponding joint part at a spaced location.

11. The implant of claim 2, wherein at least one of said first and second anchoring parts comprises a joint part mount for at least partially accommodating said respective joint part.

12. The implant of claim 11, wherein said joint part mount of one of said first and second anchoring parts comprises a surrounding edge or wall forming said at least one stop.

13. The implant of claim 1, wherein at least one of said first and second joint parts can be detachably connected with said respective first and second anchoring part.

14. The implant of claim 11, wherein said at least one joint part mount is configured to accommodate said respective joint part in a positive-locking and/or nonpositive manner.

15. The implant of claim 11, wherein said at least one joint part mount comprises a larger cross section than said respective joint part accommodated by said at least on joint part mount, said joint part mount being configured such that said joint part is displaceable on said movement surface.

16. The implant of claim 11, wherein said at least one joint part mount comprises a larger cross section than said respective joint part accommodated by said at least one joint part mount, said joint part mount being configured such that said joint part is displaceable along a straight or curved guideway on said movement surface.

17. The implant of claim 11, wherein said at least one restoring element is positioned within said at least one joint part mount in a positive-locking and/or nonpositive manner.

18. The implant of claim 2, wherein said at least one restoring element is made from a plastic material.

19. The implant of claim 2, wherein said at least one restoring element is substantially annular.

20. The implant of claim 2, characterized in that said at least one restoring element surrounds one of said first and second joint parts in a positive-locking manner.

21. The implant of claim 2, wherien said at least one restoring element comprises a leaf spring curved convexly toward at least one of said first and second joint parts.

22. The implant of claim 2, wherein said at least one restoring element is a leaf spring curved in an S-like shape.

23. The implant of claim 1, wherein said restoring device holds at least one of said first and second joint parts in said normal position in a pretensioning manner.

24. The implant of claim 2 further comprising a plurality of said restoring elements, wherein said restoring elements are arranged symmetrically around at least one of said first and second joint parts.

25. The implant of claim 1 further comprising a plurality of said restoring devices comprising different elastic properties, wherein said restoring devices are alternately positioned within said implant.

26. The implant of claim 1, wherein said first joint part comprises a concavely curved first bearing surface, and said second joint part comprises a convexly curved bearing surface.

27. The implant of claim 1, wherein said restoring device is replaceable.

28. The implant of claim 1, wherein at least one of said first and second anchoring parts is configured to be connected with a vertebral body replacement implant.

29. The implant of claim 1, wherein at least one of said first and second anchoring parts and/or said corresponding first and second joint part is comprises a wear-reducing coating forming a contact surface between at least one of said first and second anchoring parts and said corresponding first and second joint part.

30. The implant of claim 1, wherein at least one of said first and second anchoring parts and/or at least one of said first and second joint parts is made of a physiologically compatible material.

31. The implant of claim 1, wherein said first anchoring part, said second anchoring part, said first joint part, and said second joint part are made of the same physiologically compatible material or from different physiologically compatible materials.

32. The implant of claim 30, wherein said physiologically compatible material is a metal, a ceramic, or a plastic.

33. The implant of claim 31, wherein said physiologically compatible material is a metal, a ceramic, or a plastic.
